(19) United States
(12) Patent Application Publication
(10) Pub. No.: US 2023/0270534 A1
(43) Pub. Date: Aug. 31, 2023

(54) A KIT FOR VISUAL PREGNANCY DIAGNOSIS IN LIVESTOCK AND METHOD OF DETECTION THEREOF

(71) Applicant: INSTAVET BIOPHARM PVT. LTD., Maharashtra, Mumbai (IN)

(72) Inventors: Padma Venkitachalam DEVARAJAN, Mumbai (IN); Amit Sanjay LOKHANDE, Mumbai (IN); Pramod Maruti JADHAV, Dist-Kolhapur (IN)

(73) Assignee: INSTAVET BIOPHARM PVT. LTD., MAHARASHTRA,, MUMBAI (IN)

(21) Appl. No.: 18/005,788

(22) PCT Filed: Jul. 16, 2021
(86) PCT No.: PCT/IN2021/050689

§ 371 Date: Jan. 17, 2023

(30) Foreign Application Priority Data
Jul. 17, 2020 (IN) 202021002147

(51) Int. Cl. A61D 17/00

(52) U.S. Cl. CPC A61D 17/006

(57) ABSTRACT

The present invention relates to a portable kit for pregnancy detection in livestock using urine sample and its method of use. More specifically, the present invention provides a quick, simple and onsite testing kit for pregnancy estimation in livestock comprising a combination of two solutions acidifying agent and colour developing agent in a vehicle, wherein kit helps to develop a red colour in presence of Abscisic acid (about 10 μg/ml and above) as indication of pregnancy and Yellowish orange colour at lower concentration of Abscisic acid as indication of non-pregnancy. Therefore, a kit provides colour based pregnancy detection in livestock using animal urine which provides ease of testing and immediate results. The portable kit would be particularly useful in field or rural settings where laboratory equipment, chemicals, and refrigerated storage are unavailable.

### FIELD OF INVENTION

The present invention relates to a kit for colour based pregnancy detection in livestock using animal urine which provides ease of testing and immediate results. More specifically, the present invention relates to kit comprising a reagent sample composition for pregnancy estimation by visual colour detection and a portable test kit assembly and its method of use.

### BACKGROUND OF THE INVENTION

Efficient milk production farming requires that cattle be successfully bred to become pregnant 80-100 days after calving. Dairy cattle, however, have a low fertility rate with artificial insemination, requiring, on an average, 2 to 3 inseminations per conception. Therefore, a significant need exists for a method by which a dairy farmer may accurately determine that an animal is not pregnant without missing an opportunity to re-breed the animal at the next estrus period following an unsuccessful breeding. Several procedures are available, including a milk progesterone assay (Oltenacu et al., 1990; Reese et al., 2016), estrone sulfate analysis (Holdsworth et al., 1982; Warnick et al., 1995), rectal palpation (Hatzidakis et al., 1993), ultrasound (Beal et al., 1992; Cameron and Malmo, 1993), and blood tests for pregnancy-specific antigens.

Even though the prior procedures for pregnancy diagnosis are potentially useful, all have fallen short of expectations in terms of their practical, on-farm use. For example, measurements of milk or serum progesterone around day 18-22 yield unacceptably high rates of false positives (Oltenacu et al., 1990; Markusfeld et al., 1990). The presence of estrone sulfate in urine or serum provides another test, but is only useful after day 100 as concentrations rise (Holdsworth et al., 1982; Warnick et al., 1995). Transrectal ultrasonography of the uterus, using a 5 MHz linear-array or sector transducer, provides an accurate method for identifying pregnant (95 to 99%) and non-pregnant cattle (75 to 97%) post 27 days after AI under field conditions. However, Badtram et al. 1991; reported an accuracy of 69 and 72% for pregnant and non-pregnant cows, respectively, using ultrasonography between 23 and 31 days after insemination.

The discovery of pregnancy-specific protein B (PSP-B) (Butler et al., 1982) provided an approach to pregnancy diagnosis since it could be detected in the blood of pregnant cows by the fourth week of pregnancy (Sasser et al., 1986; Humblot et al., 1988).

Two other groups have developed immunoassays that may be based on an identical or immunologically similar antigen (Zoli et al., 1992a; Mialon et al., 1993; Mialon et al., 1994). In one case, the antigen (Mr-67 kDa) was called bovine pregnancy-associated glycoprotein (boPAG; now boPAG-1) (Zoli et al., 1992a); in the second, it was designated as pregnancy serum protein 60 (PSP60) (Mialon et al., 1993; Mialon et al., 1994). The immunoassay for PSP-B/boPAG1/PSP60 has two advantages. First, it can detect pregnancy relatively early. Second, interpretation of the assays does not require knowledge of the exact date of service, since boPAG-1 immunoreactive molecules are always present in the maternal serum of pregnant cows by day 28, and concentrations increase as pregnancy advances (Sasser et al., 1986; Mialon et al., 1993; Mialon et al., 1994).

There remain, however, two major disadvantages to this procedure. First, positive diagnosis in the fourth week of pregnancy remains somewhat uncertain because antigen concentrations in blood are low and somewhat variable. Second, boPAG1 concentrations rise markedly at term (Sasser et al., 1986; Zoli et al., 1992a; Mialon et al., 1993) and, due to the long circulating half-life of the molecule (Kiracofe et al., 1993), the antigen can still be detected 80-100 day postpartum (Zoli et al., 1992a; Mialon et al., 1993; Mialon et al., 1994; Kiracofe et al., 1993), compromising pregnancy diagnosis in cows bred within the early postpartum period. Thus, the test can be carried out in dairy cows at day 30, only if artificial insemination (“AI”) is performed at 45-70 days' post-partum.

Patent applications AU2002366097A1, U.S. Pat. No. 7,575,861B2 provides methods for the early detection of pregnancy in livestock such as ungulates (e.g., hoofed animals). It comprises measuring the level of at least one bovine pregnancy associated antigen (BoPAG) in the sample; and measuring the level of progesterone in the sample, wherein elevated levels of BoPAG and progesterone indicate that the bovine animal is pregnant. The sample may be from any biological specimen, including saliva, serum, blood, milk or urine. BoPAG measurement done by nucleic acid hybridization and RT-PCR. Progesterone levels measured by immunogenic detection using RIA, ELISA.

Whereas, Patent applications AU2005323518B2, AU2008243215A1 discloses methods for determining the pregnancy status of an ungulate animal by detecting the level of PAG-55 Protein fraction. These methods may involve the detection of PAG-55 enriched proteins in the PAG-55 protein fraction by using novel polyclonal and monoclonal antibodies that have been generated using the PAG-55 enriched protein fraction as an immunogen. The novel PAG-55 protein fraction indicates that the animal is pregnant.

EP2225274B1 discloses an early pregnancy test for bovine animals in which a specific polypeptide domain that is highly specific for a PAG can be detected. Method comprising: (a) contacting a sample obtained from a bovine animal with a 2D9 antibody or fragment or variant thereof, or an antibody that bind specifically to the pregnancy-associated antigens (PAGs). Unlike plate ELISA, this test does not require centrifuge for separating plasma since whole blood can be used directly in the test. The colour can be visually read. The total assay time is approximately 2 hours compared to conventional plate ELISA (4 hours).

US2008/0026384A1, US2010/0035270A1 discloses a bovine pregnancy marker is polypeptide, nucleic acid and fragment thereof. The bovine pregnancy inducible marker molecule is in SEQ ID NO1 or SEQ ID NO2. The sample is mainly blood sample. The said antibody comprises detectable label selected from the group consisting of fluoresin, rhodamine, phycoerythrin, biotin and streptavidin. The reaction product is assessed by gel electrophoresis, restriction digest mapping, scintillation counting, and filter paper assays.

U.S. Pat. No. 7,763,432 B2 discloses a purified antibody that specifically bind to pregnancy associated antigen present early in pregnancy and undetectable at about two months postpartum. Wherein the pregnancy associated antigen comprises BoPAG6 (SEQ ID NO:29). A kit comprising of suitable antibody in container which is attached to polystyrene plate, test tube or dip stick. Second antibody comprises a detectable label as fluorescent tag, biotin, a chemiluminiscent tag, or an enzyme which is alkaline phosphatase or horseradish peroxidase.

U.S. Pat. No. 8,541,187 B2 discloses a method providing a test kit including at least three test surfaces, each surface being coated with a first biomolecular recognition element specific to a marker of pregnancy. Wherein the marker is pregnancy-specific protein B (PSPB), the animal is a ruminant, and second biomolecular recognition element is a detection antibody which is retrieved from the ruminant about 3 days to about 7 days after the first sample was retrieved from the ruminant. The antibody is conjugated with a hapten. The hapten is biotin, the enzyme is horseradish peroxidase which is conjugated with streptavidin, and the visual indicator is 3,3′,5,5′-tetramethylbenzidine (TMB). Each test surface is sealed with a preserving agent derived from a blocking solution that includes a buffering agent, a non-reducing sugar, and a blocking agent, wherein the buffering agent includes NaH₂PO₄, Na₂HPO₄, and NaCl, the non-reducing sugar is sucrose, and the blocking agent is bovine serum albumin.

AU 563599B2 provides a method of detecting pregnancy at an early stage, which comprises testing a sample of a body fluid containing blood platelets in order to detect any enhanced activation of the blood platelets; enhanced activation being indicative of pregnancy. Platelet activation is detected in the presence of an inhibitor for preventing activation which is not pregnancy-specific. An activator selected from proteases, prostaglandins and adenosine diphosphate. The inhibitor is selected from heparin. anti-thrombin, Acetyl salicylic acid. Indomethacin, a combination of pyruvate kinase and phosphenol pyruvate and adenosine which comprises detecting enhanced platelet adhesion to a foreign substrate, exposing a blood sample containing an anticoagulant to the foreign substrate and detecting the number of platelets adhering to the surface; an enhancement of adherent platelets relative to a non-pregnant control indicating enhanced platelet adhesion. Platelet aggregation is monitored by a method selected from: direct observation using a microscope with phase optics, spectrophotometric densitometry and detection of the release of excretal factors from platelet granules.

All the above methods are either complex and based on PAG, Blood factors, hormone assays or rely on instrumentation and/or laboratory setup, for estimation of pregnancy in biological fluids including blood, plasma, serum, milk & urine etc. Further, some methods also mention elaborate separation techniques and multiple steps in the analysis which requires time and skilled personnel. Delay in analysis may also necessitate special storage requirements for the biological samples.

The need for simple onsite testing for pregnancy estimation is dire. The available livestock Pregnancy detection techniques pose a serious challenge of being invasive, need trained personnel, expensive, late detection (post 40 days of conception) and requirement for elaborate instrumentation and laboratory setup. Even bringing animal at veterinary clinics and handling them for such testing is difficult for the farmer, expensive and traumatic for the animal.

At present available techniques such as Rectal palpitation demands experienced trained personnel for pregnancy detection and can be performed only after 60th day of conception as early detection with this method has high abortion chances. This test is very tedious & time consuming (˜2-3h per test time) and cannot be done repeatedly, it is very painful to animal, causes bleeding in rectal area as rectal mucosa is very fragile and can cause accidental abortion. Animal may go in shock. This test is also costly (Rs.500-1000 per test). Another very important test is Ultrasonography which is though non-painful, is very costly (Rs.2000-3000 per set) and involves sophisticated instruments, trained personnel & it is time consuming too (0.5 to 2h). Even presenting animal for this test at place is very difficult due to animal's fear for such instrumentation and setup. Other method is use of detection kits which are based on detecting pregnancy associated proteins, hormones (estrone sulphate, progesterone) etc. through biological samples such as blood & blood components, & milk. But all these kits are based on ELISA testing method and are costly (USD3.5-5.5 per test) and majorly manufactured outside India. They able to detect pregnancy post 50 days of conception and demands confirmation of results after 5 consecutive days testing. There are some traditional testing methods as well such as Punyakoti Seed germination test which involves the germination of wheat soaked in cattle urine for 5 to 8 days, where no germination of seeds confirms pregnancy. This test is inaccurate, subjective, & time consuming test. Another very interesting test is to check pregnancy in cattle urine using castor oil drop formation method. In this test castor oil drops are added in urine sample and formation of one single drop of all added drops suggests pregnancy, whereas dispersed oil droplets suggests non-pregnancy. This test is also very inaccurate, lacks sensitivity, subjective and suggests pregnancy after 60th day of conception, time consuming as well.

Following mentioned are the Commercial Pregnancy test kits

### a) Using Pregnancy Associated Proteins as Markers

Biolreprod0083: A sensitive and specific double-antibody RIA for a bovine pregnancy-associated glycoprotein (bPAG) is described. The limit of detection was 0.2 ng/ml. The assay is specific for bPAG in that pituitary and placental gonadotropic hormones and other placental or serum proteins assayed. These results suggest that detection of this placental-specific antigen in the serum could be used as a specific serological method for early pregnancy diagnosis in cattle from 28 days after breeding.

The IDEXX Milk Pregnancy Test (https://www.idexx.com/en/livestock/livestock-tests/ruminant-tests/idexx-rapid-visual-pregnancy-test/), is an enzyme-linked immunoassay for the detection of pregnancy-associated glycoproteins (PAGs) in milk samples from cattle, goats, sheep and water buffaloes as a marker for pregnancy. The IDEXX Milk Pregnancy Test confirms pregnancy status from 28 days post breeding in cows and goats, from 60 days post breeding in sheep, from 29 days post breeding in water buffaloes and from 60 days post calving in cows. This enables producers to identify open animals to ensure re-breeding in a timely manner.

BioPRYN® (http://www.biotracking.com/dairy/biopryn/what) is a pregnancy detection tool for use in cattle. It measures the presence of Pregnancy-Specific Protein B (PSPB) in the blood circulation of the animal. The PSPB protein is only produced by the placenta of the growing fetus. Cows and heifers must be at least 28 days post-breeding and cows must be at least 73 days post-calving. There is a chance for a false positive if the residual PSPB has not cleared the maternal system from cows that have calved more recently than 73 days ago.

Analysis of BoPAGs in particular has exhibited potential for use in pregnancy testing. However, such tests can yield high false positive rates. This error rate occurs because the PAG test is done at day 25 of pregnancy. However, some embryos die between day 20 and 25 of pregnancy. This dying tissue can probably produce some PAG. Thus, the cow is PAG positive, but the embryo is dead. The results of this can be a false positive rate of 8%, which is generally considered to be unacceptable within commercial breeding programs.

### b) Other Commercial Products Using Hormonal Measurements

EMLAB GENETICS LLC, P-TEST (https://www.emlabgenetics.com/p-test) measures the pregnancy hormone, estrone sulfate, using a simple and rapid colorimetric reaction. In cattle, pregnancy can be detected as early as 60 days from breeding. P-TEST can easily detect pregnancy in cattle, sheep, goats, pigs, deer, elk, bison, llamas, alpaca, and camel. The accuracy in predicting pregnancy is 97.4%. The accuracy of predicting non-pregnancy is 94.4%. The sensitivity of this test is >95%. It takes 10 min to predict results. But this test requires urine temperature to be matched the room temperature before testing and shows good detection post 2^{nd} and 3^{rd} trimester of pregnancy.

Cow progesterone colloidal gold test strip (http://www.livestocktool.com/product/cattle-farming-equipments)/veterinary-instruments-for-cattle/cow-pregnancy-test.html). This is the urine pregnancy test kit. This kit can determine pregnancy based on progesterone content. It's a Cattle pregnancy test strip (paper based) used for early pregnancy diagnosis, with high accuracy, easy to operate, rapid detection, require room temperature storage, easy to carry, harmless to the mother and foetus. But the progesterone concentration in urine sample for early pregnancy detection (˜18-22 days post AI) gives high rates of false positives.

The problem associated with available test methods and kits clearly indicates the dire need for a simple yet innovative, on site use, cost effective, sensitive, accurate, rapid and non-invasive technique for pregnancy diagnosis in livestock which could be helpful for farmers to recognize pregnancy at the earliest opportunity so as to plan the breeding of the livestock at the very next opportunity.

Therefore, a need for pregnancy tests that could be carried out reliably and early in pregnancy with improved accuracy. A pregnancy test could provide definitive indication as to whether rebreeding or culling is required. In general, AI is successful less than 50% of the time and the producer must either rely on overt signs of return to estrus (that are easily missed) or delay rebreeding until pregnancy failure is confirmed by one of the methods described above. Such delays are extremely costly and constitute a major economic loss to the industry. These methods would be particularly useful in field or rural settings where laboratory equipment, chemicals, and refrigerated storage are unavailable. There is thus a need for a feasible, sensitive and accurate pregnancy test in livestock that yields a low level of false positive results.

The present inventors have arrived at a kit for visual detection of Pregnancy in biological fluids urine for rapid and on site detection using a simple process steps to use the said kit that provides two different colours which relate to conditions of pregnant & non pregnant livestock using urine samples which has immense value. Further being based on simple visual detection this method obviates the need for any kind of instrumentation or specialized trained personnel. Therefore, the present invention of the kit for visual detection of livestock pregnancy status in urine sample provides onsite, point of care detection method with great advantages for humans and livestock. The benefit of this early pregnancy detection is that identifying animals that are not pregnant very shortly after breeding, allows for timely rebreeding and minimizes the amount of time the animal is open.

### OBJECTIVES OF THE INVENTION

The primary objective of present invention is to provide a simple, rapid on site kit for visual pregnancy detection in livestock urine.
One more objective of the present invention is to provide a simple affordable portable kit for visual pregnancy detection in Urine with great outreach.
Another objective of the present invention is to provide a method of onsite pregnancy detection in urine sample.
Yet another objective is to screen for low and high levels of Abscisic acid in urine based on visual colour detection.

### SUMMARY OF THE INVENTION

In accordance to present invention, it provides a stable and economic kit for detection of Abscisic acid in urine sample of livestock as indication of pregnancy, comprising of:
plurality of transparent tubes for collection of urine sample from subject animal,
a first solution of acidifying agent,
a second solution of parahydroxy benzaldehyde as colour developing agent in a vehicle in concentration range between 0.4 to 1% w/v,
Separate droppers with graduations for addition of first and second solutions,
standard colour bands as indication for pregnancy detection;
Characterised in that the kit provides a colour development in urine sample solution in presence of first solution and second solution, wherein the colour is red colour in presence of Abscisic acid about 10 μg/ml and above as indication of pregnancy as validated by traditional testing methods and Yellowish orange colour at lower Abscisic acid concentrations is an indication of non-pregnancy.

Wherein the tube is selected from glass tube and plastic tube.

Wherein, the vehicle is preferably sulfuric acid having normality between 30 N to 36.8 N sulfuric acid (H₂SO₄) solution.

In accordance to second embodiment, the acidifying agent is selected from hydrochloric acid, sulfuric acid, phosphoric acid, nitric acid, acetic acid, trichloroacetic acid and mixtures thereof. The concentration of acidifying agent in water is between 0.1 to 36.8 N solution.

In accordance to one more embodiment, the present invention provides a method for colour development for detection of Abscisic acid in urine sample as indication of livestock pregnancy using kit disclosed in present invention, the said method comprising of steps:
Step 1—Collection of livestock urine sample in empty tubes,
Step 2—Addition of 0.1 to 0.5 mL of first solution of acidifying agent using graduated dropper and mixing,
Step 3—Addition of 0.2 to 0.5 mL of second solution of colour developing agent in vehicle using graduated dropper and mixing to develop the colour solution,
Step 4—determining the developed colour with standard colour bands to detect the concentration of Abscisic acid in urine sample for indication of pregnancy in said livestock, wherein development of red colour indicates pregnancy and yellowish orange colour indicates non pregnancy.

### BRIEF DESCRIPTION OF THE DRAWING

Further aspects and advantages of the present invention will be readily understood from the following detailed description with reference to the accompanying figures of the drawings. The figures together with a detailed description below, are incorporated in and form part of the specification, and serve to further illustrate the embodiments and explain various principles and advantages but not limiting the scope of the invention. In the accompanying drawings:

FIG. 1: Illustrates an image of colour development a) before and b) after Interference removal from Urine sample upon acid pre-treatment as per present invention.

FIG. 2: Illustrates colour bands a) positive control for Abscisic acid about 10 μg/ml and above as indication of pregnancy and b) negative control for lower concentration of Abscisic acid as indication of non-pregnancy.

FIG. 3: Illustrate an image of colour development with respect to Effect of different acidifying agent on detection of pregnancy as per present invention kit.

### DETAILED DESCRIPTION OF THE INVENTION

The present invention relates to a visual kit for pregnancy detection in livestock using urine sample, its composition and its method of use for Abscisic acid (ABA) level estimation in urine sample. The said Pregnancy detection kit comprises of a specific reagent composition A and B. To a specific amount of urine when Reagent A and B are added sequentially a visual colour change is instantaneously observed based on the amount of Abscisic acid present. A red colour in presence of Abscisic acid about 10 μg/ml and above as indication of pregnancy and Yellowish orange colour with lower amount of Abscisic acid as indication of non-pregnancy, which is then matched with the provided standard colour bands.

In the present invention, the kit for visual detection of livestock pregnancy status comprises Reagent A, Reagent B, glass/plastic transparent tubes and calibrated droppers and standard colour bands to match the colour generated in the samples. The method is rapid, based on simple visual detection, cost effective and can be performed at the livestock/animal side providing major advantage.

The reagent sample mentioned above is composed of two parts, A] an acidic composition of acids (Normality: 0.1N to 36.8N) such as hydrochloric acid, sulfuric acid, phosphoric acid, nitric acid, acetic acid, etc. and B] a reagent solution of ABA specific dye such as parahydroxy benzaldehyde (PHB) in sulfuric acid solution (0.4 to 1% w/v) (Normality: 30 to 36.8N).

Accordingly, the present invention relates to a kit for visual detection of livestock pregnancy in urine sample, which provides a practical and cost effective approach more specifically the advantages of portable reagent test kit assembly of present invention, coupled with its simple on site method of use make the invention economically viable with very high commercial application.

The present invention therefore provides kit for visual detection of livestock pregnancy status in urine samples on site. The said pregnancy detection kit offers an advantage such as simple portable kit, simple method of use, suitable combination of materials and tools, no withdrawal of blood sample, no biological sample preprocessing, rapid, visual colour based pregnancy estimation, affordable and thus has great outreach. The ease of handling and estimation is facilitated by kit design and its method of use based on minimum and cost effective materials and tools. This invention also offers a major advantage of minimal cost and early on site rapid visual pregnancy detection.

The present invention furthermore discloses a kit for visual detection of livestock pregnancy status in urine samples onsite which can overcome limitations of current estimation such as cost, travel, inconvenience and time. Detection can be done in farm settings where commonly used facilities for early, rapid, pregnancy detection is not available and also in hospitals, dispensaries, rural clinics, veterinary field. Thus, it provides a simple, economic yet effective solution to the existing lacuna.

Accordingly, kit for visual detection of livestock pregnancy status comprises, (i) Reagent A in a tube comprising of acidic composition of acids (Normality: 0.1 N to 36.8 N) such as hydrochloric acid, sulfuric acid, phosphoric acid, acetic acid etc. (ii) Reagent B in tube comprising of ABA specific reagent parahydroxy benzaldehyde (PHB) in the concentration of 0.4 to 1% w/v in sulfuric acid (Normality: 30 N to 36.8 N), (iii) Empty tubes for collecting urine samples (iv) Calibrated Dropper for transferring Reagent A & Reagent B sequentially in sample tube (vi) Standard colour bands for visual comparison.

In accordance to second embodiment, the present invention provides a method to detect the pregnancy using said kit. The method of use of the kit for visual detection of livestock pregnancy status involves the following steps
Step 1—Collection of livestock urine sample in empty tubes
Step 2—Addition of specific amount of reagent [A] into sample tube with the help of provided calibrated dropper and mixing
Step 3—Addition of reagent B into sample tube with the help of provided calibrated dropper and mixing
Step 4—Compare the developed colour with standard colour bands.

Wherein, a red colour develops in presence of Abscisic acid about 10 μg/ml and above as indication of pregnancy and Yellowish orange colour develops at lower concentration of Abscisic acid as indication of non-pregnancy, which is then matched with the provided standard colour bands.

The foregoing description of the invention has been set merely to illustrate the current stage of invention and is not intended to be limiting. Since further development on detection techniques such as to record the reading in digital form/image form or introducing/collaborating the available techniques for minimizing the scope of errors or maximizing the detection and sensitivity of the disclosed embodiments is possible. Therefore, such modification will towards incorporating the spirit and substance of the invention may occur to person skilled in the art, the invention should be construed to include everything within the scope of the disclosure.

The present invention is further illustrated in connection with particular examples as follows.

### Example 1: Reaction of Abscisic Acid with Colour Developing Agent Parahydroxy Benzaladehyde (PHB)

A. Preparation of Abscisic acid (ABA) standards: Standard ABA was dissolved in distilled water and following concentrations were made. A=Blank, B=10 μg/mL, C=0.2 μg/mL and D=0.02 μg/mL.

B. Preparation of Para-hydroxy Benzaldehyde (PHB) Reagent (Second regent): About 100 mg of PHB was dissolved in 15 ml of Sulfuric acid (30 to 36.8 N)

Reaction: To the 0.4 ml of blank solution three ABA standard samples 0.6 ml of PHB reagent was added and observed for colour development.

Result: Increased concentration of ABA leads to development of red colour in the sample (Tube A-pinkish, Tube B-pinkish red/red, Tube C-Pink and Tube D-pinkish).

### Example 2: Optimization of p-Hydroxybenzaldehyde (PHB) Concentration (i.e. Second Solution of Colour Developing Agent in a Vehicle)

The different concentrations of PHB was prepared in varied concentration of Sulfuric acid (H₂SO₄) (30 to 36.8 N). The concentration of PHB was ranging from 1 mg to 10 mg/0.6 mL (about 0.1 to 2% w/v) of Sulfuric acid (H₂SO₄) (30 to 36.8 N). Then 0.6 mL of this reagent was added to the 0.4 mL of standard ABA samples corresponding to Pregnant (ABA, 10 μg/mL) & Non-pregnant (ABA, 0.02m/mL) concentrations of ABA in cattle urine.

Result: It was found that 3 to 5 mg PHB in 0.6 mL of H₂SO₄ (30 to 36.8 N) showed a good colour discrimination between different ABA concentrations corresponding to low and high concentration of ABA. Therefore, decided to use this concentration of PHB 3 to 5 mg/0.6 ml of Sulfuric acid (30 to 36.8 N) to develop the kit of present invention.

### Example 3: Experiments Using Cattle Urine Sample

A. Detection of Abscisic acid present in Cattle Urine: To the 0.4 mL of cattle urine samples (pregnant & non-pregnant), 0.6 mL of PHB in sulphuric acid (30 to 36.8 N) was added and observed for colour difference.

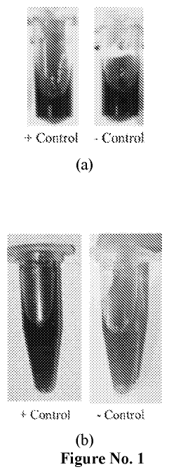

Result: Dark reddish brown colour was developed in all samples and difficult to distinguish between pregnant (+Control) and non-pregnant urine (−Control) samples as can be seen in FIG. 1a.

As cattle urine contains many components that might interfere with the PHB solution, there was need to remove such interfering substances.

B. Removal of Interference using acidifying agent pre-treatment (need of first solution): Urine samples were pre-treated with acidifying agent H₂SO₄ (0.1 to 36.8 N) to remove interference and observed better discrimination. These pre-treated urine samples were then reacted with PHB solution & were assessed for colour development in pregnant and non-pregnant cattle urine samples.

Result: A confirm visual colour difference was observed between pregnant (+Control) and non-pregnant (−Control) cattle urine (FIG. 1b).
Reason for adding Acidifying agent in Reagent A (first solution): The acid addition removes interference associated with Urine samples and provides better colour discrimination between Pregnant and non-pregnant samples

Any Acid could be added as an Acidifying agent and can be selected from HCl, H₂SO₄, HNO₃, Phosphoric acid, Acetic acid, Trichloroacetic acid and etc in the normality range of 0.1 to 36.8 N.

C. Optimization for Reagent A (first solution) and Reagent B (second solution) volumes:
a) In first case, acidifying agent Reagent A—H₂SO₄ (0.1 to 36.8 N) was kept constant 0.4 mL and the amount of Reagent B [PHB in H₂SO₄; 4 mg/0.6 mL (30 to 36.8N)] was varied from 0.1 to 0.5 mL. Colour difference was analysed.
b) In second case, the amount of Reagent B (PHB in H₂SO₄; 4 mg/0.6 mL) was kept constant as 0.3 mL and the amount of acidifying agent Reagent A (H₂SO₄; 0.1 to 36.8 N) was varied from 0.1 mL to 0.5 mL. Again, colour difference for final optimization was analysed.

Result: From both the variations, it was observed that at 0.1 to 0.5 mL of acidifying agent Reagent A H₂SO₄ (0.1 to 36.8 N) and 0.2 to 0.5 mL of Reagent B [PHB in H₂SO₄; 4 mg/0.6 mL (30 to 36.8 N)] showed good colour difference between pregnant and non-pregnant cattle urine. Hence, used these optimized amounts of reagents for point of care test kit preparation for pregnancy detection in cattle.

### Example 4: A Performance of Pregnancy Test Kit as Per Present Invention

A. Method for using Kit and its Protocol: Pregnant and non-pregnant cattle urine samples were tested as per the kit procedure mentioned below.

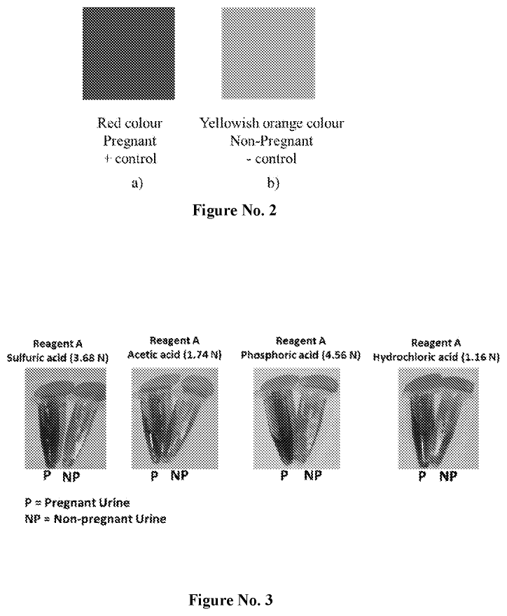

To the 1 ml Cattle Urine sample added 0.2 ml of first solution Reagent A (sulfuric acid 0.1 to 36.8 N) followed by 0.3 ml of second solution Reagent B [PHB in H₂SO₄; 4 mg/0.6 mL (30 to 36.8 N)] and developed colour was observed and matched with standard color bands as shown in FIG. 2, red colour indicates pregnancy (FIG. 2a) and yellowish orange colour indicates non-pregnancy (FIG. 2b)

Result: The pregnant urine sample developed a red colour (+control) and non-pregnant urine sample developed a yellowish orange colour (−Control) as can see in FIG. 1b. The test procedure was distinguishing between both the samples.

### Example 5: Effect of Different Acids as Vehicle for PHB Colour Developing Agent

Effect of different acids as PHB vehicles was evaluated. PHB was added 100 mg in 15 ml of all acids separately and mixed for 15 min. The reagent was observed for uniform mixing and appearance.

Result: Only concentrated acids dissolved PHB. The results are depicted in below Table 1.

TABLE 1
Effect of using different acids as vehicle for PHB reagent

| | Acid | PHB | | |
|---|---|---|---|---|
| | volume | amount | | |
| Name of Acid | (ml) | (mg) | Observation | Remark |
| Conc HNO₃ | 15 | 100 | Dissolved | Not suitable |
| | | | completely but | for kit use |
| | | | generates Strong | |
| | | | irritating fumes | |
| Conc HCl | 15 | 100 | Not dissolved | Not suitable |
| | | | completely, | for kit use |
| | | | generates strong | |
| | | | irritating fumes | |
| Conc H₂SO₄ | 15 | 100 | Dissolves PHB and | Suitable for |
| | | | no fumes generation | Kit use |
| Phosphoric | 15 | 100 | PHB Not dissolved | Not suitable |
| acid | | | completely | |
| Trichloroacetic | 15 | 100 | PHB Not dissolved | Not Suitable |
| acid | | | completely | |
| Acetic acid | 15 | 100 | PHB Not dissolved | Not suitable |
| glacial | | | completely | |

Reason for adding PHB in Acid vehicle: PHB reacts with ABA and this reaction is acid catalysed. Acid dilution with water affects this reaction and loses interpretation thus normality of the acid must be at 30 to 36.8 N.

### Example 6: Effect of Different Acidifying Agent: It can be Selected from any Acids HCl, HNO₃, H₂SO₄, Phosphoric Acid, Acetic Acid, and Trichloroacetic Acid as Acidifying Agent in the Normality Range of 0.1 to 36.8 N

Wherein, 10% v/v of different acids were used as Reagent A first solution and added to 1 ml of cattle urine sample (pregnant & non pregnant).

Then Reagent B i.e. PHB in sulphuric acid H₂SO₄ (30 to 36.8 N) was added to develop a colour: The results are depicted in below FIG. 3 and Table 3. Any acid in the normality range of 0.1 to 36.8 N can provide discrimination between pregnant and non-pregnant cattle urine samples.

TABLE 2
Normality of different acids

| | Normality of | Normality 10% |
|---|---|---|
| | concentrated | v/v diluted |
| Name of Acid | solution | solution |
| HNO₃ | 15.8 | 1.58 |
| HCl | 11.65 | 1.16 |
| H₂SO₄ | 36.8 | 3.68 |
| Phosphoric acid | 45.6 | 4.56 |
| Trichloroacetic acid (20% w/v) | 1.22 | 0.12 |
| Acetic acid glacial | 17.4 | 1.74 |

TABLE 3
Colour developed in samples with use of different acidifying agent.

| | Colour with Urine | Colour with Urine |
|---|---|---|
| Acidifying agent | sample of | sample of non- |
| (10% v/v) used | Pregnant cattle | Pregnant cattle |
| H₂SO₄ | Red | Yellowish orange |
| Acetic acid glacial | Red | Yellowish orange |
| Phosphoric acid | Red | Yellowish orange |
| Hydrochloric acid | Red | Yellowish orange |

### Example 7: Stability Data for Kit—the Stability of Reagent was Assessed for 3 Months at 2-8° C., 25° C., and 40° C. the Reagent was Evaluated for Discriminating Colour Development when Reacted with 1 ml of Pregnant and Non-Pregnant Cattle Urine Samples as Per the Kit Procedure

Result: Stability is depicted in Table 4 as correct colour development observed for both samples (i.e. a red colour in Pregnant cattle urine and yellowish orange colour in Non-Pregnant Urine samples). The reagent was found stable at all storage conditions and is able to discriminate between Pregnant and Non-Pregnant Urine samples.

TABLE 4
Stability of PHB reagent

| Period in Months | 2 to 8° C. | 25° C. | 40° C. |
|---|---|---|---|
| 1 month | Stable | Stable | Stable |
| 2 months | Stable | Stable | Stable |
| 3 months | Stable | Stable | Stable |

I claim:

1. A kit for colour development for detection of Abscisic acid in urine sample as indication of livestock pregnancy, comprising of:
a transparent tube for collection of urine sample from subject animal,
a first solution of acidifying agent,
a second solution of colour developing agent in a vehicle,
droppers with graduations for addition of first and second solutions,
a standard colour bands
Characterised in that the kit provides a colour development in urine sample solution in presence of first solution and second solution, wherein the colour is red colour in presence of Abscisic acid (about 10 μg/ml and above) as indication of pregnancy and Yellowish orange colour at lower concentration of Abscisic acid as indication of non-pregnancy.

2. The kit as claimed in claim 1, wherein the tube is selected from glass tube and plastic tube.

3. The kit as claimed in claim 1, wherein colour developing agent is selected from parahydroxy benzaldehyde.

4. The kit as claimed in claim 1, wherein the vehicle is sulfuric acid.

5. The kit as claimed in claim 4, wherein normality of sulfuric acid vehicle is selected between 30 N to 36.8 N.

6. The kit as claimed in claim 1, wherein acidifying agent is selected from hydrochloric acid, sulfuric acid, phosphoric acid, nitric acid, acetic acid, trichloroacetic acid and mixtures thereof.

7. The kit as claimed in claim 1, wherein in the second solution concentration of colour developing agent in vehicle is between 0.4 to 1% w/v.

8. The kit as claimed in claim 1, wherein concentration of first solution acidifying agent in water is between 0.1 to 36.8 N solution.

9. A method for colour development for detection of Abscisic acid in urine sample as indication of livestock pregnancy using kit as claimed in claim 1, comprising of:
Step 1—Collection of livestock urine sample in empty tubes,
Step 2—Addition of 0.1 to 0.5 mL of first solution of acidifying agent using graduated dropper and mixing,
Step 3—Addition of 0.2 to 0.5 mL of second solution of colour developing agent in vehicle using graduated dropper and mixing to develop the colour solution,
Step 4—Comparing the developed colour with standard colour bands to detect the concentration of Abscisic acid in urine sample for indication of pregnancy in said livestock, wherein development of red colour indicates pregnancy and Yellowish orange colour indicates non pregnancy.
